(19) United States
(12) Patent Application Publication
(10) Pub. No.: US 2004/0127471 A1
(43) Pub. Date: Jul. 1, 2004

(54) Methods of treating age associated memory impairment (AAMI), mild cognitive impairment (MCI), and dementias with cell cycle inhibitors

(76) Inventor: Barry Reisberg, New York, NY (US)

Correspondence Address: KLAUBER & JACKSON, 411 HACKENSACK AVENUE, HACKENSACK, NJ, 07601

(21) Appl. No.: 10/664,817

(22) Filed: Sep. 17, 2003

Related U.S. Application Data
(60) Provisional application No. 60/411,282, filed on Sep. 17, 2002.

(51) Int. Cl. A61K 31/65; A61K 31/60; A61K 31/404; A61K 31/353; A61K 31/19

(52) U.S. Cl. 514/165; 514/456; 514/414; 514/557; 514/152

(57) ABSTRACT

Therapeutic methods for treatment of age associated memory impairment (AAMI), mild cognitive impairment (MCI), Alzheimer's disease (AD), cerebrovascular dementia (CVD), and related neurodegenerative conditions by administering an agent capable of inhibiting cell cycle progression, comprising administering one or more agents that are capable of inhibiting neuronal cell cycle progression at either an early cell cycle phase or generally, either alone or in combination with one or more agents capable of reducing mitogenic stimulation.

### FIELD OF THE INVENTION

The present invention relates to methods for the treatment of retrogenic conditions and disorders, including, for example, age associated memory impairment (AAMI), mild cognitive impairment (MCI), Alzheimer's disease and cerebrovascular dementia, using compounds that inhibit cell cycle progression alone or in combination with an agent or agents that enhance the efficacy of cell cycle inhibitors.

### BACKGROUND OF THE INVENTION

Normal cell division occurs through a molecular biologic process known as the cell cycle. The cell cycle consists of major phases known as the G₁ phase, the S phase, the G₂ phase, the M phase and the G₀phase. These phases of cell division correspond to the early growth phase, the synthesis phase, a later growth phase, a mitosis phase, and a resting phase. Progression through these phases is regulated by a series of enzymes, which include activators and inhibitors. Some of these cell cycle enzymes and factors have been related to the development of neurofibrillary changes in AD, and consequently, the neurofibrillary tangles which are characteristic of AD. Activation of these cell cycle enzymes and factors therefore in some cases appears to precede the development of neuronal neurofibrillary changes (Reisberg et al. (2002a) Am. J. Alzheimer's Dis. 17:202-212; Nagy (2000) Neurobiol. Aging 21: 761-769; Busser et al. (1998) J. Neuroscience 18: 2801-2807). Since the clinical symptoms of mild cognitive impairment (MCI) and progressive AD are accompanied by progressive neurofibrillary changes (Reisberg et al. (2000) In: World Psychiatric Association Series: Evidence and Experience in Psychiatry, Vol. 3 Dementia (Maj & Sartorius, eds.), Chichester; John Wiley & Sons; pp. 69-115; de Leon et al. (2002) Neuroscience Lett.), the activation of these cell cycle enzymes and factors, therefore, in some cases precedes the development of MCI and AD. The cell cycle division time in lymphocyte cultures in AD patients and aged controls in comparison with normal young adults has been studied (Fischman, Reisberg, et al. (1984) Biological Psychiatry 19:319-327). The cell cycle was found to be 50% longer both in AD patients and in the aged controls than in normal young adults. Recent studies have indicated that enzymes representative of various phases of the cell cycle are stimulated in neurons in Alzheimer's disease (AD), cerebrovascular dementia (CVD) and other dementias (Reisberg et al. (2002a) Am. J. Alzheimer's Dis. 17:202-212).

AD pathology is associated with the development of β-amyloid plaques, which appears to result from an attempt of the neuron to regenerate (Wu et al. (2000) Neurobiology Aging 21:797-806; Lee et al. (2002) Nature 405:360-364). The stimulation to regenerate, i.e., divide, is evidenced by activation of various mitogenic (cell cycle) markers, including the mitogen-activated protein kinase (MAPK) cascade, cyclins and cyclin-dependant kinases. The activation of these mitogenic factors has been shown in some cases to precede the development of neuronal neurofibrillary changes (Nagy et al. (2000) Neurobiology Aging 21:761-769; Busser et al. (1998) J. Neuroscience 18:2801-2807). Neurofibrillary changes are associated with the neurofibrillary tangles which form inside neurons, and are one of the pathologic hallmarks of AD.

Some of the activated mitogenic factors have been related to the phosphorylation and hyperphosphorylation of the tau protein (see, for example, Patrick et al. (1999) Nature 402:615-622), a major constituent of AD neurofibrillary tangles. Hyperphosphorylation of tau promotes neurofibrillary tangle development. Thus, the cell cycle factors which are activated appear to promote AD neurofibrillary pathology by promoting tau hyperphosphorylation. Reactivation of the mitogenic factors has also been related to amyloid protein precursor processing into amyloidogenic elements (Suzuki et al. (1994) EMBO J. 13:1114-1122).

### SUMMARY OF THE INVENTION

The present invention is based, in part, on the realization that entry of the neuron into the cell cycle promotes neurological and psychiatric conditions, disorders and diseases, including age associated memory impairment (AAMI), mild cognitive impairment (MCI), Alzheimer's disease (AD), and cerebrovascular dementia (CVD). The present invention is further based, in part, on the realization that retrogenic conditions, disorders, and dementias, such as AAMI, MCI, AD, and CVD can be treated with a cell cycle inhibitor that has its effect at an early phase of the cell cycle, e.g. before the S phase of the cell cycle, to prevent neuronal apoptosis, since apoptosis occurs after the S phase of the cell cycle has been initiated.

Accordingly, in a first aspect, the invention provides a method of treating AAMI, MCI, Alzheimer's disease, CVD and related dementias, comprising administering a therapeutically effective amount of at least one agent capable of inhibiting neuronal cell cycle progression to a subject diagnosed as having the retrogenic condition, disorder, or dementia.

In a second aspect, the method of the invention comprises administering at least one agent that inhibits cell cycle progression prior to entry of a neuronal cell into a synthesis S phase.

In a third aspect, the method of the invention comprises administering a therapeutically effective amount of at least one agent capable of inhibiting neuronal cell cycle progression at or before the early growth (G₁) phase.

In a fourth aspect, the invention provides a method of treating age associated memory impairment (AAMI), mild cognitive impairment (MCI), Alzheimer's disease (AD), cerebrovascular dementia (CVD), and related degenerative disease in a subject having the condition, disorder, or degenerative disease, comprising administering a therapeutically effective amount of (i) at least one first agent capable of inhibiting neuronal cell cycle progression, and (ii) at least one second agent capable of reducing mitogenic stimulation, wherein the first agent inhibits cell progression prior to entry of a neuronal cell into the synthesis (S) phase, and the second agent is capable of reducing mitogenic stimulation at any phase of the cell cycle.

In a fifth aspect, the invention provides a method of treating a subject diagnosed as having AAMI, MCI, Alzheimer's disease, CVD, or related degenerative diseases or at risk of having AAMI, MCI, Alzheimer's disease, CVD, or related degenerative diseases comprising administering a therapeutically effective amount of at least one agent capable of inhibiting neuronal cell cycle progression at the early growth (G₁) phase to the subject, either alone or in combination with at least one second agent capable of inhibiting cell cycle progression at any phase of the cell cycle.

In a sixth aspect, the invention provides a method of treating age associated memory impairment (AAMI), mild cognitive impairment (MCI), Alzheimer's disease (AD), cerebrovascular dementia (CVD), or related degenerative diseases in a subject with the symptoms of, or at risk for, AAMI, MCI, AD, CVD, or related degenerative diseases comprising administering a therapeutically effective amount of (i) at least one first agent capable of inhibiting cell cycle progression, and (ii) at least one second agent capable of reducing mitogenic stimulation, wherein the first agent inhibits cell progression prior to entry of a neuronal cell into a synthesis (S) phase and the second agent is capable of reducing extracellular and/or intracellular mitogenic stimulation such as glutamate-induced excitotoxicity and/or activated microglia-induced mitogenic stimulation.

In a seventh aspect, the invention provides a method of treating age associated memory impairment (AAMI), mild cognitive impairment (MCI), Alzheimer's disease (AD), cerebrovascular dementia (CVD) or related degenerative diseases, in a subject with the symptoms of, or at risk for, AAMI, MCI, AD, CVD, or related degenerative diseases, comprising administering a cocktail, or drug combination, that includes a therapeutically effective amount of: i) a first agent or agents which are capable of inhibiting neuronal cell cycle progression at one or more phases of the cell cycle, that may, but is not required to, include or be limited to, an early phase of the cell cycle; and ii) a second agent or agents capable of reducing mitogenic stimulation, where the mitogenic stimulation is either glutamate-induced and/or activated microglia-induced excitotoxicity.

These and other aspects of the present invention will be better appreciated by reference to the following Detailed Description.

### DETAILED DESCRIPTION OF THE INVENTION

Before the present methods and compositions are described, it is to be understood that this invention is not limited to particular methods and compositions, as such methods and compounds may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “an inhibitor of extracellular mitogenic stimulation” includes mixtures of such inhibitors, reference to “the formulation” or “the method” includes one or more formulations, methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

Definitions

The phrase “retrogenesis” or “retrogenic processes”, etc., is a term used to describe the observation that degenerative biological processes reverse the order of acquisition of the same mechanisms in normal development. A retrogenic disease, disorder, or condition is a neurological or psychiatric disorder associated with a subjectively and/or objectively perceived loss of neuropsychiatric capacity, including cognitive, functional, and/or neurologic capacity.

The phrase “early phase” refers to a point in mitosis that occurs prior to the synthesis (S) phase. Normal cell division occurs through a cell cycle consisting of major phases known as the early growth phase (G₁), the synthesis phase (S), a later growth phase (G₂), a mitosis phase (M), and a resting phase (G₀).

The phrase “an inhibitor of early growth (G₁) phase” and the like, means an agent or compound capable of inhibiting neuronal cell cycle division at the early growth (G₁) phase.

The phrase “extracellular mitogenic stimulation” refers broadly to factors and conditions which work collectively with other factors and conditions to send a neuronal cell through the cell cycle in the direction of cell division. The mitogenic stimulation factors and conditions include both intracellular and extracellular factors. These factors encompass those that result in excitotoxicity (e.g., glutamate-induced excitotoxicity), or mitogenic factors resulting from activated microglia.

The phrase “an inhibitor of mitogenic stimulation” means an agent or compound capable of reducing mitogenic stimulation, including glutamate-induced excitotoxicity, or activated microglia-related mitotic stimulation. Examples of such compounds include, but are not limited to memantine, neramexane, amantadine, riluzole, MK801, ketamine, dextromethorphan, dextrorphan, phencyclidine, dexanabinol (HU-211), and anti-inflammatory agents such as non-steroidal anti-inflammatory compounds (NSAIDS) (e.g., ibuprofen, naproxen, celecoxib, rofecoxib, sulindac, piroxicam, indomethacin, etodolac, nabumetone, tolmetin, diclofenac, ketoprofen, apazone, meloxicam), salicylates such as acetylsalicylic acid, steroids such as glucocorticoids (e.g. prednisone), and immunophillins (e.g., cyclosporin A, tacrolimus).

By the term “treatment” is meant the administration of medicine to ameliorate AAMI, MCI, Alzheimer's disease, CVD or a related neurodegenerative condition in a patient suffering from such a condition or affliction. Amelioration of the condition includes slowing the progression of the process and/or disease, arresting the progression of the process and/or disease, or reversing the progression of the process and/or the disease. A specific aspect of the instant invention is the identification of treatment populations at risk for a neurodegenerative condition prior to development of a neurodegenerative condition, e.g. prior to development of MCI, Alzheimer's disease or cerebrovascular dementia. As described more fully below, the method of the invention treats subjects identified at an early stage of neurological impairment, including for example, age associated memory impairment (AAMI) and mild cognitive impairment (MCI), and prior to the development of more advanced diseases such as, for example, Alzheimer's disease (AD), and cerebrovascular dementia (CVD).

By the term “related retrogenic diseases”, “related dementias”, “related neurological conditions and disorders”, and the like, is meant neurological conditions that present some degree of degeneration from the normal adult neurological condition. This includes measurable decline of normal cognitive, neurologic, or functional capacity. Decline in normal neurological capacity can be determined by any method known to the art, including the global deterioration scale (GDS) (Reisberg, et al. (1982) Am. J. Psychiatry 139: 1136-1139) and/or the functional assessment staging (FAST) procedure (Reisberg (1988) Psychopharmacology Bulletin 24: 653-659).

A “therapeutically effective amount” is an amount of a reagent sufficient to achieve the desired treatment effect. In this case the desired effect of the therapeutically effective amount of the reagent would be an amount sufficient to slow or reduce the progression, or reverse some of the symptoms of, AAMI, MCI, Alzheimer's disease, CVD or a related neurodegenerative condition in a patient suffering from such a condition or affliction.

General Aspects of the Invention

More than two decades of studies of the clinical nature of normal aging and Alzheimer's disease (AD) based upon detailed, systematic observations have provided the inventor with the basis for concluding that the successive functional stages and substages in the progression of normal aging and AD are a precise reversal of the order of acquisition of the same functions in normal human development (Reisberg et al. (1986a) Geriatrics 41:30-46; Reisberg et al. (1986b) In: Biological Psychiatry, Vol. 7, C. Shagass et al. (Eds.). New York: Elsevier Science Publishing Co. pp. 1319-1321). Present, previously unpublished data from the inventor's research, described below, provides overwhelming support for this observation. The levels of functional loss in the dementia of Alzheimer's disease, as well as in related dementias, and extending (in reverse order) into MCI and AAMI are shown in the following table. Also shown is the approximate corresponding developmental age (DA) of acquisition of the same functions in the course of normal human development.

TABLE 1
LEVELS OF FUNTIONAL LOSS IN ALZHEIMER'S
DISEASE (AD) WITH CORRESPONDING DEVELOPMENTAL
AGES (DA) OF FUNCTIONAL ACQUISITION*

| | Functional | | |
|---|---|---|---|
| | Assessment | | |
| | Staging | | APPROXIMATE |
| LEVEL OF FUNCTIONAL | (FAST) | | DA OF FUNCTION |
| LOSS IN AD | LEVEL | | ACQUISITION* |
| Inability to hold up head | 7 | f | 4 to 12 weeks |
| independently | | | |
| Inability to smile | 7 | e | 8 to 16 weeks |
| Inability to sit up | 7 | d | 6 to 9 months |
| independently | | | |
| Inability to walk | 7 | c | 12 months |
| independently | | | |
| Inability to voluntarily | 7 | b | 12 months |
| say more than a single | | | |
| intelligible word in | | | |
| response to queries | | | |
| Inability to voluntarily | 7 | a | 15 months |
| say more than about a half- | | | |
| dozen intelligible words | | | |
| in response to queries | | | |
| Inability to maintain bowel | 6 | e | 24 to 36 months |
| control | | | |
| Inability to maintain | 6 | d | 36 to 54 months |
| bladder control | | | |
| Inability to independently | 6 | c | 48 months |
| perform mechanics of | | | |
| toileting correctly | | | |
| Inability to bathe properly | 6 | b | 4 years |
| without assistance | | | |
| Inability to dress (put on | 6 | a | 5 years |
| clothing) properly without | | | |
| assistance | | | |
| Inability to independently | 5 | | 5 to 7 years |
| select proper attire for | | | |
| the occasion and the season | | | |
| Inability to perform complex | 4 | | 8 to 12 years |
| activities of daily life | | | |
| independently (e.g., managing | | | |
| finances, planning and | | | |
| preparing a meal for guests, | | | |
| marketing) | | | |
| Inability to perform with | 3 | | Adolescence |
| customary facility in | | | |
| demanding employment and | | | |
| social settings | | | |
| Subjective deficit in adult | 2 | | Adult |
| functional capacity | | | |
| Neither subjective nor | 1 | | Adult |
| objective deficit in | | | |
| functional capacities | | | |

*Adapted from Reisberg, B. Dementia: A systematic approach to identifying reversible causes. Geriatrics 1986; 41 (4): 30-46

Clearly, these stages of AD and related dementias, MCI, and AAMI appear to remarkably reverse those of normal human development. The validity of these stages was examined in cross-sectional study of 1507 subjects in comparison with cognition based (non-functional) Mini Mental State Examination (MMSE) (Folstein et al. (1975) J Psychiatr Res 12:189-198) scores. The study population consisted of 1507 participants in ongoing longitudinal studies of aging and dementia. At baseline evaluation, community residing participants received extensive cognitive, psychiatric, physical, and neurologic examinations, an electrocardiogram, complete blood and urine laboratory screening, a neuroimaging study of the brain, and evaluation with a comprehensive psychometric screening battery. Strict exclusion criteria were applied at baseline, in order to eliminate subjects with confounding factors that can cause loss of functional and cognitive capacity. Cognitive, psychometric, and medical follow-up evaluations were conducted for selected ambulatory participants at 2-year intervals, and full re-evaluations were conducted for all eligible participants at 4 to 5 year intervals. Follow-up evaluations were conducted in the research center setting or, when necessary, in the patient's residential home or in the institution where the patient resides, and when this was not possible, by telephone interview. Data regarding co-morbid conditions were obtained from the medical history as provided by the participant or a knowledgeable informant, as well as from physical and neurological examinations, and from medical and nursing home records. For the present study, the latest evaluation of the subject, was utilized. For all baseline and follow-up evaluations used for this study, subjects with conditions other than AD that can cause loss of function, were excluded. All subjects in this study had a diagnosis of either normal aged (GDS stage 1); AAMI (GDS stage 2), MCI (GDS stage 3); or Alzheimer's type dementia in accordance with the DSM IV criteria for a diagnosis of dementia and the NINCDS-ADRDA criteria for probable AD (American Psychiatric Association (1994) Diagnostic and Statistical Manual of Mental Disorders (4^{th} Edition), Washington, D.C. American Psychiatric Association; McKhann, et al. (1984) 34; 939-944.

Degree of cognitive impairment was assessed with the MMSE, a standard dementia screening instrument. The MMSE has no criteria for functioning and was not designed to differentially diagnose presence or absence of subjective memory deficit. Patients with severe dementia invariably reach bottom score levels on the MMSE. Functional status was measured with the FAST procedure. Based on empirical studies of functioning in aging and dementia, the FAST describes an ordinal hierarchic sequential pattern of functional decline, from normal aging to severe stage AD in seven stages and eleven substages, constituting sixteen successive levels of incremental functional loss. The sixteen functional FAST levels correspond with landmarks of normal functional attainment in infancy and childhood (Reisberg, B. (1986) Geriatrics 41 (4): 30-46) (Table 1).The FAST encompasses a full range of functional complexity, including high-level executive functions; skills for independent survival in community settings, also called “instrumental activities of daily life” (IADL); routine daily self-care functions, commonly called “activities of daily life” (ADL); and the more basic physical functions of bladder and bowel control, verbalization and ambulation. FAST scores of 1 or 2 indicate respectively no or only subjective functional deficit, a FAST score of 3 indicates functional impairment which is most consistent with a diagnosis of MCI, and a FAST score of 4 and greater indicates functional deficit sufficiently severe so as to be compatible with a diagnosis of dementia. The FAST scale points are scored at the level of the highest sequential (ordinal) deficit. Functional deficits resulting from evident physical co-morbidity commonly occur out of sequence, and receive a separate “non-ordinal” scoring in addition to the ordinal FAST score. The ordinal FAST score determines AD related disability; the non-ordinal FAST score indicates excess disability. The FAST is scored on the basis of information obtained from knowledgeable proxies and also from direct personal observation by the examiner. Unlike the MMSE, the FAST does not screen cognition in the patient and is thus potentially independent of the MMSE and other cognitive assessments.

The eleven FAST substages were combined into four FAST levels for the analysis: FAST substages 6a, 6,b and 6c into one FAST level “deficiency in ADL”; FAST substages 6d and 6e into one FAST level “incipient incontinence”; FAST sub-stages 7a and 7b into one FAST level “incipient averbal”; and FAST substages 7c,7d,7e and 7f into one FAST level “immobile”, resulting in a total of 9 ordinal FAST levels.

Pearson correlation was used to determine the relationship between MMSE scores and the nine ordinal numerical FAST levels, i.e., FAST stages 1, 2, 3, 4, 5, and FAST substage combinatorial levels 6abc, 6cd, 7ab, and 7cdef. ANOVA with Tukey HSD procedure with MMSE as the independent variable was used to determine the significance of FAST level differences. Pearson correlation was also used to determine the relationship between mean MMSE scores and the sixteen ordinally enumerated FAST stages and substages. Partial correlation was used to determine influence of age, gender, educational attainment, and co-morbid conditions on the relationship between MMSE scores and the sixteen FAST stages and substages.

There were 704 baseline evaluations and 803 follow-up evaluations. There were 376 participants (25 percent) with no or only subjective memory deficit; 243 participants (16 percent) with a diagnosis of MCI, and 888 patients (59 percent) with a diagnosis of probable AD according to DSM IV and NINCDS-ADRDA criteria. Of the 450 participants with a FAST score of 1 and 2, a majority of 369 (82 percent) had no or only subjective cognitive impairment; 79 (17.6 percent) had MCI; and 2 (0.4 percent) had AD. Of the171 participants with a FAST score of 3, a majority of 142 (83 percent) had MCI; 5 (3 percent) had no or only subjective cognitive impairment; and 24 (14 percent) had early AD. All but 24 (2.7 percent) of the 886 participants with FAST scores of 4 through 7f had AD, 22 (2.5 percent) of the remaining subjects had MCI, and 2 of the remaining subjects had AD.

Each of the nine consecutive FAST levels within the assessment range of the MMSE was associated with a decrease in mean MMSE score. A strong linear relationship was observed between these two potentially independent assessments (r=0.90, p<0.0001). The strength of this linear relationship remained essentially unchanged after controlling for age and education (r=0.87, p<0.0001), and for gender (r=0.89, p<0.0001). ANOVA showed a significant overall difference in mean MMSE score (p<0.0001) between the nine individual FAST levels. Tukey HSD procedure with MMSE as independent variable showed most individual FAST levels to be statistically significantly different (p=<0.0001) from each other in terms of mean MMSE score with two exceptions: no statistically significant difference existed in terms of mean MMSE score between FAST levels 1 and 2 (p>0.05), probably because the MMSE was not designed to differentially diagnose aged persons without subjective deficits (FAST level 1) from persons with subjective deficits (FAST level 2). Also, no statistically significant differences were noted between consecutive FAST stages 7ab and 7cdef (p>0.05), ostensibly because of bottoming out of the MMSE.

In conclusion, these findings indicate that in the pathologic process that leads from normal aging to MCI and advancing AD, progression and degree of cognitive decline are strongly associated with incrementally progressing losses of developmental landmark functions. The course of this conjoint cognitive and functional decline reverses the conjoint course of cognitive and functional human development. Potentially confounding factors that can influence functioning, i.e., age, education, gender, and co-morbid conditions did not notably influence the FAST-MMSE relationship. It should also be noted, that the FAST levels comprise a hierarchy of functions, from most complex to least complex, which are equivalent to the normative functional landmarks of human development. The outcome of this study supports the retrogenetic concept of AD as a conjoint cognitive and functional regression to ever earlier developmental stages.

The relationship between normal human development and AD has been termed “retrogenesis”, and is defined as a process by which degenerative biological mechanisms reverse the order of acquisition of the same mechanisms in normal development (Reisberg et al. (1999a) Intl. Psychogeriatrics 11:7-23 and (1999b) Eur. Arch. Psychiatry Clin. Neuroscience 249 (Suppl. 3):28-36). The retrogenic processes relates in part to myelin changes which occur in the brain of the patient with normal aging, AAMI, MCI, AD, CVD and related retrogenic dementias. This process is also related to recent findings regarding the molecular biology of normal cellular development and the changes in these normal molecular processes in AD, CVD and other retrogenic dementias (Reisberg et al. (2002) Neurobiology Aging 23(1S):S555) and (2002 a) Am. J. Alzheimer's Dis 17:202-212).

The instant invention is based in part on the realization that the molecular mechanisms associated with the reactivation of the cell cycle in neurons account in part for the retrogenic process in AD, CVD and other dementias in which the retrogenic process occurs. The most metabolically active regions of the brain in AD, are the areas that are the most capable of responding to a mitogenic stimulus, and these are the most vulnerable in AD. Thus, these molecular processes appear to account for the neurologic, cognitive, functional, and other retrogenic phenomena observed in AD.

In 1986, a new diagnostic entity termed age associated memory impairment (AAMI) was proposed (Reisberg, et al., 1986 Developmental Neuropsychology 2: 401-412). Other current terms for this entity include age associated cognitive decline (Levy, (1994) International Psychogeriatrics 6(1): 63-68), age related cognitive decline (APA (1994) Diagnostic and Statistical Manual of Mental Disorders, (₄^{th} Ed.) Washington, D.C.: American Psychiatric Association) and age associated cognitive impairment, among others. The new entity was based on the global deterioration scale (GDS) (Reisberg, et al. (1982) Am. J. Psychiatry 139: 1136-1139) which differentiated the aging process and progressive degenerative dementia in seven major stages. The first stage of the GDS is one in which individuals at any age have neither subjective complaints of cognitive impairment nor objective evidence of impairment. These GDS stage 1 individuals are considered normal. The second stage of the GDS applies to those generally elderly persons who complain of memory and cognitive functioning difficulties such as not recalling names as well as they could five or ten years previously or not recalling where they have placed things as well as they could five or ten years previously. These subjective complaints appear to be very common in otherwise normal elderly individuals (Lowenthal, et al., (1967) Aging and Mental Disorder in San Francisco: A Social Psychiatric Study. San Francisco: Jossey Bass; Sluss, et al. (1980) Gerontologist (Part II) 20:201 (Abstract); Lane & Snowdon (1989) In Lovibond & Wilson (Eds.), Clinical and Abnormal Psychology, pp. 365-376, North-Holland: Elsevier; Reinikainen, et al. (1990) Neurology 40 (Supp;.1): 177; Larrabee & Crook (1994) International Psychogeriatrics 6:95-104; Wang, et al. (2000) J. Am. Geriatric Soc. 48: 295-299). The term “age associated memory impairment” (AAMI) has been proposed for elderly persons in GDS stage 2. Subsequent research has indicated that GDS stage 2 (AAMI) elderly persons differ neurophysiologically from elderly persons who are normal and free of subjective complaints, i.e., GDS stage 1. For example, AAMI subjects have been found to have more electrophysiologic slowing on a computer analyzed EEG than GDS stage 1 elderly persons (Prichep, John, Ferris, Reisberg, et al.(1994) Neurobiol. Aging 15: 85-90). More recent research now indicates that AAMI individuals do progress to the next stage of MCI at a rate of approximately 7 to 12% of AAMI subjects per year (de Leon, Convit, Wolf, Tarshish, DeSanti, Rusinek, Tsui, Kandil, Scherer, Roche, Imossi, Thorn, Bobinski, Caraos, Lesbre, Schlyer, Poirier, Reisberg, and Fowler, (2001) Proc. Natl. Acad. Sci. USA 98: 10966-10971; Reisberg, et al. (2002c, d) In: Principles and Practice of Geriatric Psychiatry, 2^{nd} Ed. (Copeland et al., eds.) Chichester (UK): John Wiley, pp. 142-145 and 308-312). Present unpublished data from the inventor's longitudinal studies now provides unprecedented evidence for the differentiation of the AAMI entity associated with GDS stage 2 (subjective complaints) from elderly subjects who are free of subjective complaints and therefore classified in GDS stage 1. A consecutive series of healthy subjects with normal aging seen for a longitudinal research investigation were examined. Subjects who fulfilled the criteria seen at baseline between 1984 and 1997 were selected. There were 453 cases whose initial diagnosis was normal aged or AAMI (GDS stage 1 or 2) in the period from 1984-1997. Of these there were 179 subjects who were at GDS stage 1 (normal aged) at baseline and 274 were at GDS stage 2 (AAMI) at baseline. Only 9 of the 179 normal aged GDS stage 1 subjects had MCI or dementia at follow-up. In contrast, 112 of the aged subjects with a baseline at GDS stage 2 (AAMI) had MCI or dementia at follow-up. Since there were so few of the GDS=1 group who went on to MCI or dementia, only the GDS=2, AAMI, group was used in the analysis. There were also 77 subjects in the GDS=2 baseline AAMI group who had only a single visit, who were therefore dropped from the analysis (two subjects were dropped for other reasons), giving a final N of 197.

Variables used in analyses consisted of 2 sets:

1) A combinatorial psychometric variable known as the psychometric deterioration score (PDS) (Reisberg, et al., (1988) Drug Dev. Res. 15; 101-114), Hamilton depression scale (Hamilton, M. (1960) Journal of Neurology, Neurosurgery and Psychiatry 23: 56-62), total score (items 1-21), sex, age at initial visit, and total of Brief Cognitive Rating Scale (BCRS) items 1-5 (Reisberg & Ferris, (1988) Psychopharmacology Bulletin 24: 629-636).

2) Hamilton items 1-21, all PDS items, sex, age at initial visit, and total of BCRS items 1-5.

The individual BCRS items 1-5 and the BEHAVE-AD items (Reisberg et al.(1987) J Clin Psychiatry 48 [5,Suppl]: 9-15), were entered in analyses and were not significant.

Analyses were performed on the individuals who had AAMI (GDS Stage 2) at baseline to explore the relationship between their background variables and the length of time to conversion. Because the time to convert is both left censored (the actual date of conversion is not known) and right censored (an individual might convert at a date later than the last visit), a Weibull model of survival was used (SAS procedure LWFEREG) to analyze time to conversion. The patient variables considered were the variables HDT21 (total, 21 item Hamilton depression scale scores) and PDS. The original model fit also contained gender, age at baseline, and the sum of BCRS items 1-5, but these variables were dropped since they were not significant.

TABLE 2

| | | | | | | | Probability of converting by | | |
|---|---|---|---|---|---|---|---|---|---|
| Quartile | | Quartile | | Survival_Distribution | | | 2 | 5 | 10 |
| HDT21 | | PDS | | Q75 | Q50 | Q25 | years | years | years |
| * L | 1 | L | 1.3 | 6.6 | 11.8 | 18.6 | .05 | .17 | .42 |
| L | 1 | M | 2.0 | 4.7 | 8.3 | 13.1 | .08 | .27 | .60 |
| L | 1 | H | 3.0 | 2.8 | 5.0 | 8.0 | .16 | .50 | .86 |
| M | 3 | L | 1.3 | 5.8 | 10.4 | 16.4 | .06 | .20 | .48 |
| M | 3 | M | 2.0 | 4.1 | 7.3 | 11.6 | .09 | .32 | .67 |
| M | 3 | H | 3.0 | 2.5 | 4.5 | 7.0 | .19 | .56 | .91 |
| H | 7 | L | 1.3 | 4.5 | 8.1 | 12.8 | .08 | .28 | .62 |
| H | 7 | M | 2.0 | 3.2 | 5.7 | 9.0 | .13 | .43 | .80 |
| H | 7 | H | 3.0 | 1.9 | 3.5 | 5.5 | .26 | .70 | .97 |
| Mean | 4.8 | | 2.1 | 3.5 | 6.2 | 9.8 | .12 | .39 | .76 |

The Table 2 displays for various quartiles of HDT21 and PDS, the 25^{th}, 50^{th} and 75^{th} quartiles of the fitted survival distributions of time to conversion to MCI or dementia (GDS≧3) from AAMI (GDS stage 2), as well as the probability of conversion at 2, 5 and 10 years obtained from the same fitted survival distribution. For example, among patients whose baseline values for HDT21 and PDS are in the lowest respective sample quartiles, 75% have not converted by 6.6 years, 50% have not converted by 11.8 years and 25% have not converted by 18.6 years. For these same patients 5% convert by 2 years, 17% convert by 5 years and 42% convert by 10 years.

The equation below represents the Weibull survival distribution fit, S(t). For any time T (in years) the equation yields the percentage 100p % of the population that will not have converted given the values of HDT21 and PDS at baseline.

S(t)=e^{(-exp[(Log(T)-(9.3181-0.0623HDT21-0.4988PDS))/0.6581])}

For the same Weibull survival distribution fit, the next equation gives for any probability p, the time T (in years) at which 100p % of the population will not have converted given the values of HDT21 and PDS at baseline.

T=e^{(0.6581Log (-Log(p))+9.3181-0.0623HDT21-0.4988PDS)/}365.25

Note: Log is the natural Log function

These results are very important. First, these results indicate that psychometric tests and/or a psychometric test battery can be useful in predicting decline in AAMI subjects. Second, these results also indicate that mood variables are also significant predictors of decline in AAMI elderly persons. Clearly, these results also point to the mean duration of AAMI as well as variables influencing the mean duration. This mean duration, i.e. the point at which subjects with median psychometric and Hamilton mood scores demonstrate a 50% likelihood of progression is 7.3 years.

This rate of progression is consistent with a duration of AAMI of approximately 15 years. The cause of AAMI symptomatology is presumed to be related to the neuropathologic changes which are seen in normal elderly individuals including neurofibrillary tangles and senile plaques comprised in part of β-amyloid. This neuropathology in normal aging is the same as that of AD, except that in AD there are greater quantities of these neuropathologic changes in relevant brain regions. Accordingly, treatment of AAMI subjects with cell cycle inhibitors is expected to result in therapeutic benefits in preventing further pathologic changes and forestalling the development of more severe clinical, as well as pathologic conditions such as MCI, AD and other related dementias.

The term “mild cognitive impairment” (MCI) has been proposed (Reisberg et al. (1988) Drug Develop. Res. 15: 101-114; Flicker, Ferris, and Reisberg, (1991) Neurology 41: 1006-1009) for the condition in which there are subtle, clinically manifest deficits in cognition, memory, and functioning, amongst other impairments which are observed which are not of sufficient magnitude to fulfill criteria for an AD (McKhann, et al. (1984) Neurology 34: 939-944) or other dementia diagnosis. Studies indicated that persons with MCI commonly develop dementia, especially AD, over the next few to several years (Flicker et al. (1991) supra; Kluger, Ferris, Golomb, Mittelman, and Reisberg (1999) J. Geriatric Psych. Neurol 12: 168-179). MCI subjects have greater hippocampal atrophy on neuroimaging than AAMI and normal aged subjects (de Leon, George, Golomb, Tarshish, Convit, Kluger, DeSanti, McRae, Ferris, Reisberg, et al. (1997) Neurobiol. Aging 18: 1-11; De Santi et al. (2001) Neurobiol. Aging 22: 529-539). MCI subjects also have significantly greater psychometric test deficits (Reisberg, et al. (1988) supra), balance and coordination deficits (Franssen, Souren, Torossian, and Reisberg (1999) J. Am. Geriatric Soc. 47: 463-499), and deficits on motor performance tasks (Kluger, Gianutsos, Golomb, Ferris, George, Franssen, and Reisberg (1997a) J. Gerontology: Psychol. Sci. 52B: P28-P39; Kluger, Gianutsos, Golomb, Ferris, and Reisberg (1997b) International Psychogeriatrics 9 (Suppl.1): 307-316) than AAMI and normal aged subjects. Because MCI subjects commonly develop AD and other dementias, it is clear that this pathology is on a continuum with that of AD and related dementias. Further evidence for this continuum is the continuum of hippocampal neuropathologic change which has been observed (de Leon et al. (1997) supra; De Santi et al. (2001) supra). New data on the continuum of neuropathologic change from normal aging and AAMI subjects to MCI, comes from recent CSF marker studies, which indicate that markers for neurofibrillary pathology (Ptau23 1) and β-amyloid pathology (AP40 and AP42), increase in MCI subjects in comparison with normal aging and AAMI subjects (de Leon et al. (2002) Neuroscience Lett.). This evidence further provides support for the therapeutic use of cell cycle inhibitors in treating MCI as well as in preventing and/or postponing AD and other related dementias in MCI patients.

Vascular brain abnormalities commonly coexist with the neuropathologic features of AD. Furthermore, studies have shown that these pathologies, i.e., AD pathology and cerebrovascular disease pathology interact synergistically (Jagust (2001) Lancet 358: 2097-2098). A common pathologic process can be described which appears to explain the commonly observed similarity of the clinical symptomatology in CVD and AD and the interaction of these conditions which commonly occurs. CVD is also commonly termed vascular dementia and has been recently referred to as vascular brain burden. This process of vulnerability has been termed “arborial entropy” (Reisberg, et al. (2002e) In: Vascular Cognitive Impairment, Enkinjuntti & Gauthier, eds. Martin Dunitz, London; pp. 557-569). The myelin cover appears to provide protection to the axon, and the most thinly myelinated brain regions are the most vulnerable in AD and CVD. The AD pathogenic process and processes which produce CVD both attack the myelin. The response of the brain to metabolic stressors such as anoxia, which has been associated with CVD, is an attempt of neurons to regenerate, and this attempt to regenerate is expressed as reactivation of mitogenic/cell cycle factors in neurons (Smith et al. (1999) Neuroscience Lett. 271: 45-48; Husseman et al. (2000) Neurobiol. Aging 21: 815-828; Arendt (2001) Neuroscience 102: 723-765). Therefore, inhibition of cell cycle reactivation is expected to be effective in treating CVD, as well as AD.

A review of the literature reveals that generalized cell cycle inhibitors cause cognitive impairment. The cognitive impairment associated with generalized cell cycle inhibitors appears to result from the apoptosis which is triggered by reentry of neurons into the cell cycle. Mature neurons have long been known as unique cells in the adult, in that they were believed to be incapable of cell division. However, more recently it has been discovered that, although the majority of adult brain neurons are incapable of mitosis, some cell division does occur. This division is by far the exception and the vast majority of adult brain neurons are incapable of mitosis (cell division). The reentry of maturely differentiated neurons into the cell cycle produces an apoptotic response which results in programmed cell death (Liu et al. (2001) Cell Tissue Res. 305:217-228; Qin et al. (1994) Proc. Natl. Acad. Sci. USA 1991:10918-10922). Neuronal apoptosis has been shown in cell culture models to be accompanied by changes in cell cycle enzymes (Park et al. (1997a) J. Neuroscience 17:8975-8983; Park et al. (1997) J. Neuroscience 17:1256-1270; Park et al. (1998a) J. Cell Biol. 143:457-467; Park et al. (1998b) J. Neuroscience 18:830-840; Padmanabhan et al. (1999) J. Neuroscience 19:8747-8756; O'Hare et al. (2000) J. Biol. Chem. 275:25358-25364).

The association of generalized cell cycle inhibitors with cognitive impairment is seen, for example, in learning disabilities associated with treatment of children with methotrexate (Yanovski et al. (1989) Med. Pediar. Oncol. 17:216-221). Children who received central nervous system chemotherapy were found to experience greater difficulty in academic achievement and other neurocognitive deficits (Brown et al. (1999) J. Dev. Behav. Pediatr. 5:373-377). Breast carcinoma patients treated with a combination of cyclophosphamide, flurouracil, and methotrexate chemotherapy had a significantly higher risk of late cognitive impairment (Schagen et al. (1990) Cancer 85:640-650).

Retrogenic processes occur as a result of cell cycle activation produced by stressors, which result in neuronal injury and ultimately, programmed cell death. To treat AAMI, MCI, AD, CVD and other retrogenic dementias, agents must be administered which prevent neuronal injury before inevitable cell injury and cell death occur. Since apoptosis occurs after the S phase of the cell cycle has been initiated and is well underway, cell cycle inhibitors that work earlier in the cell cycle are required for the treatment of AAMI, MCI, AD, CVD and other retrogenic dementias. Thus, compounds that inhibit the cell cycle before the S phase are particularly useful for the treatment of AAMI, MCI, AD, CVD and related retrogenic dementias.

### DETAILED DESCRIPTION OF PREFERRED EMBODIMENTS

The invention provides methods for the treatment of a subject with, or at risk for, AAMI, MCI, AD, CVD and other retrogenic dementias, by administering an agent or combination of agents capable of inhibiting neuronal cell cycle progression. A single agent could be used, but in a more likely scenario, a cocktail, or combination of therapeutic agents may be used. Such combinations include, but are not limited to the following:

i) one or more first agents, where the first agent is capable of inhibiting neuronal cell cycle progression at an early phase, a phase other than an early phase, or generally in more than one phase of the cell cycle; and optionally

i) one or more second agents, where the agent may be any or all of a number of agents capable of inhibiting mitogenic stimulation either by inhibiting glutamate-induced excitotoxicity and/or activated microglia-induced mitogenic stimulation. Preferred choices for each of the aforementioned agents are described below.

Cell Cycle Inhibitors

In one embodiment of the method of the invention, the therapeutic agent is minocycline, also known as Minocin, Minocin IV, Vectrin, and Dynasin. More generally, the agent may be any tertacycline family derivative that is capable of crossing the blood-brain barrier. The cell cycle inhibitor may also be acetylsalicyclic acid, known as aspirin, or any salicylate that is capable of inhibiting the early phase of the cell cycle. The agent may be sirolimus, also known as rapamune or rapamycin, or any derivative of rapamycin capable of inhibiting the cell cycle, flavopiridol, ciclopirox, a paulone, indirubin, fascaplysin, olomoucine, roscovitine, Aragusterol A, valproate (also known as valproate sodium, Depacon, Depakene, or valproic acid), N-(3-chloro-7-indolyl)-1,4-benzenedisulfamide (E7070), or a farnesyl transferase inhibitor such as R115777, SCH66336 and BMS-214662, or sodium butyrate.

Early Phase Inhibitors

In another embodiment of the invention, the therapeutic agent is an early phase cell cycle inhibitor. Preferably the agent is minocycline, also known as Minocin, Minocin IV, Vectrin, and Dynasin. More generally, the agent may be any tertacycline family derivative that is capable of crossing the blood-brain barrier. The cell cycle inhibitor may also be acetylsalicyclic acid, known as aspirin, or any salicylate that is capable of inhibiting the early phase of the cell cyle. The agent may be sirolimus, also known as rapamune or rapamycin, a paulone, indirubin, fascaplysin, olomoucine, roscovitine, aragusterol A, valproate (also known as valproate sodium, Depacon, Depakene, or valproic acid), N-(3-chloro-7-indolyl)-1,4-benzenedisulfamide (E7070), farnesyl transferase inhibitors R115777, SCH66336 and BMS - 214662, or sodium butyrate.

Minocycline (Minocin, Minocin IV, Vectrin, Dynacin ) is a semisynthetic second generation tetracycline compound. Minocycline has long been used as an antimicrobial agent and is presently approved in the US for the treatment of acne vulgaris, gonorrhea, syphilis, mycobacterium marinum and for the treatment of organisms with demonstrated sensitivity to the compound (Physician's Desk Reference (PDR) 57 h Edition, 2003, pp. 3420-3422, 3422-3424, 1921-1923, 3270-3272).

Previous studies have found that the tetracycline family of compounds, of which minocycline is a member, have various actions apart from their antimicrobial effects (Golob et al. (1998) Adv. Dent. Res. 12:12-26; Ryan and Ashley (1998) Adv. Dent. Res. 12:149-151). Importantly, minocycline has been shown to inhibit nitric oxide (NO) induced p38 MAP Kinase phosphorylation (Ghatan et al. (2000) J. Cell Biol. 150:335-347; Lin et al. (2001) Neuroscience Letters 315:61-64). The mitogen-activated protein (MAP) kinases are a family of serine/threonine kinases that are mediators of the entry and progress of the cell through each of the phases of the cell cycle. The p38 MAP kinase pathway mediates the regulation of cyclin D expression. Cyclin D is a mediator of the induction of the cell cycle from the G₀ (resting) state to the G₁ (initial growth [mitotic] stage) (Nagy (2000) Neurobiology of Aging 21: 761-769). The inventor concludes that minocycline can inhibit entry into the cell cycle. These effects on the cell cycle likely account in part for other observed effects of minocycline including the recently uncovered effects on cerebral ischemia and inflammation (Yrjanheikki, et al. (1999) Proc. Nat). Acad. Sci. USA 96:13496-13500; Yrjanheikki, et al. (1998) Proc. Natl. Acd. Sci. USA 95:15769-15774). These effects of minocycline include inhibition of nitric oxide induced neuronal death by inhibition of nitric oxide induced activation of p38 MAP kinase (Lin, et al., (2001) Neuroscience Letters 315:61-64).

Since p38 MAP kinase activity has been implicated in nitric oxide induced apoptosis and apoptosis occurs from re-entry of neurons into the cell cycle past the G₁/S check point, the inventor concludes that minocycline is enhancing neuronal survival by this cell cycle modulatory effect. Specifically, under developmental conditions, nitric oxide is associated with growth arrest and inhibition of entry into the cell cycle G_{I} and S phase (Nagy (2000) Neurobiology of Aging 21: 761-769; Arendt (2000) Neurobiology of Aging 21: 783-796). However, in Alzheimer's disease and related dementias, nitric oxide activates p21 ras which results in cellular activation (Lander et al., (1995) J. Biol. Chem. 270:7017-7020). It has been noted that nitric oxide synthetase and p21 ras expression in neurons vulnerable to neurofibrillary degeneration in AD provides the basis for a mechanism that might enhance the progression of neurodegeneration in AD (Arendt (2000) Neurobiology of Aging 21: 783-796).

Consequently, minocycline in inhibiting nitric oxide toxicity in the AD context, and in the context of other retrogenic processes, is different from that in the context of normal development and minocycline inhibits early cell cycle progression in AAMI, MCI, AD, CVD and other retrogenic dementias. Since minocycline crosses the blood brain barrier and achieves brain concentrations in the rat of 30 to 40% those of the systemic exposure, minocycline is effective on these processes through various routes of administration (Colovic and Caccia (2003), Journal of Chromatography B 791:337-343).

Acetylsalicylic acid (aspirin) is a versatile and valuable therapeutic agent which is indicated for the treatment of fever (antipyretic), mild pain (analgesic), the prevention of myocardial infarction, the prevention of strokes and transient ischemic attacks, and the treatment of arthritis, among many other conditions. Presently, aspirin is widely used as well for the prevention of cancer (Giovannucci et al. (1995) New Engl. J. Med. 333:609-614). There is evidence for the therapeutic utility of aspirin in lung, colon and heart cancers, among others (Rosenberg, et al. (1991) J. Natl. Cancer Inst. 83: 355-358; Schreinemachers and Everson (1994) Epidemiology 5:138-146; Pelage, et al. (1994) Arch. Intern. Med. 154: 394-399). The mechanism of action of aspirin's therapeutic effects in neoplastic diseases (cancers) remains controversial.

Aspirin and related salicylates have varied and sometimes contradictory effects on the cell cycle. For example, in terms of the p38 mitogen activated protein kinase pathway described above for minocycline efficacy, sodium salicylate has been found to have an apparently opposite effect from that of minocycline, although salicylates inhibit the early phase cell cycle response simultaneously through effects on other pathways (Schwenger (1997) Proc Natl Acad Sci U.S.A. 94:2869-2873; Vartiainen (2003) Stroke 34:752-757).

Specifically, the pathway through which aspirin and related salicylates inhibit the early phase cell cycle response is the growth factor stimulated mitogen activated protein kinase pathway (p42/p44 MAPK). This pathway also results in the stimulation of cyclin D expression and activation of cdk4 and cdk6. The result of this activation cascade is the mitogenic activation of cells and entry from the G₀ to the G₁ phase. The p44 and p42 MAPKs are synonymously termed extracellular signal regulated kinases, ERK-1 and ERK-2, respectively. Apart from but probably related to, the mitogenic stimulatory effect, ERK-2 has been implicated in the hyperphosphorylation of tau and paired helical filament formation in in-vitro studies (Drews et al. (1992) EMBO J 11:2131-2138; Goedert et al (1992) FEBS Lett 312:95-99). Aspirin has been shown to inhibit ERK-1 and ERK-2 (p44 and p42 MAPK) activation in response to hypoxia/reoxygenation injury (Vartiainen, et al. (2003) Stroke 34:752-757). The inventor concludes that in at least one important pathway, aspirin and related salicylates can inhibit early phase cell cycle entry, serve as a mitogenic inhibitor and prevent the development of retrogenic disorders including AAMI, MCI, AD, CVD and other retrogenic dementias. A further conclusion is that aspirin and related salicylates can be synergistic with minocycline in preventing retrogenic disorders.

Sirolimus, also known as rapamune or rapamycin, is a compound generally classified as an immunophillin. Other immunophillins include cyclosporin A and tacrolimus (Prograf, FK 506). It has been found that the immunophillins are enriched 10-50 times in the central nervous system as compared with tissues of the immune system (Steiner et al. (1997) Nature Medicine 3:421-428).

Sirolimus has been previously found to markedly increased neurite outgrowth in PC12 cells in adrenal gland tumors (PC12 cells) in the presence of a low concentration of nerve growth factor, whereas tacrolimus had little effect on neurite outgrowth, even in the presence of nerve growth factor (Parker et al. (2000) Neuropharmacology 39:1913-1919). Unlike tacrolimus, sirolimus is known to inhibit cellular proliferation at the GI phase of the cell cycle (Dumont et al. (1996) Life Science 58:373-395; Thomas et al. (1997) Current Opinion Cell Biology 9:782-787; Brunn et al. (1997) Science 277:99-101). Further, sirolimus prolongs cell cycle progression in the mid to late G₁ phase (Terada et al. (1993) Clin. Biochemistry 154:7-15; Sehgal (1998) Clin. Biochemistry 31:335-340). More specifically, Terada et al. (1995) J. Immunol. 155:3418-3426 proposed that the inhibition of ribosomal protein synthesis by sirolimus results in the prolongation of the G₁ phase. Other work has found that sirolimus markedly reduces the kinase activity of the cdk 4/cyclin D and cdk 2/cyclin E complexes which peak in the mid to late portions of the G₁ phase (Wood et al. (1994) Perspec. Drug Disc. Design 2:163-185; Sherr (1994) Cell 79:551-555).

From the foregoing it may be concluded that sirolimus as an early phase cell cycle inhibitor and a mitogenic inhibitor can prevent the development of mitogenic disorders including AAMI, MCI, AD, CVD, and other retrogenic dementias. A further conclusion is that sirolimus can be used synergistically with minocycline and related tetracycline derivatives and/or with aspirin and related salicylates.

These biomolecular effects of sirolimus can potentially translate into overt and clinically relevant effects on somatic tissues. For example, it has been found that sirolimus inhibits the proliferation of vascular smooth muscle cells through the ₁/S transition (Marx et al. (1995) Circulation Res. 76:412-417). An effort has recently been made to translate these effects of sirolimus into therapy for percutaneous transluminal coronary angioplasty (PTCA) restenosis by inhibiting cellular proliferation (Poon et al. (2002) Lancet 359:619-622).

Paulones and indirubin inhibit cyclin dependent kinases and cell cycle progression (Zaharevitz, et al. (1999) Cancer Res. 59:2566-2569; Hoessel, et al. (1999) Nature Cell Biol. 1:60-67). These compounds are of utility in blocking cell cycle progression and thereby inhibiting the retrogenesis process in AAMI, MCI, AD, CVD and other retrogenic dementias.

Fascaplysin is a naturally occurring substance which has been shown to be an early phase cell cycle inhibitor. Fascaplysin has been shown to specifically inhibit cdk4 (Soni, et al. (2000) Biochemical and Biophysical Research Communications 275: 877-884). Cdk4 is a key kinase which interacts with cyclin DI and is involved in the entry of the cell into the cell cycle from G₀ (quiescence) and in the G₁/S transition. Fascaplysin, as an early phase cell cycle inhibitor is of utility in inhibiting the retrogenic process in AAMI, MCI, AD, CVD and other retrogenic dementias.

Olomoucine and roscovitine (R-roscovitine, CYC 202) have been shown to cause cell cycle arrest at the G₁ phase ( Alessi, et al. (1998) Experimental Cell Research 245:8-18). These compounds inhibit cdk2 activity and stop or delay entry into the S phse of the cell cycle (Alessi, et al. (1998) Experimental Cell Research 245:8-18; McClue et al. (2002) Int. J. Cancer 102:463-468; Schutte, et al. (1997) Experimental Cell Research 236: 4-15). Consequently these compounds are of utility in blocking cell cycle progression and inhibiting the retrogenic process in AAMI, MCI, AD, CVD and other retrogenic dementias.

Aragusterol A (YTA0040) is a steroid which is derived from a sponge of the genus Xestospongia. This compound has been shown to possess anti-tumor activity. Studies have demonstrated that YTA0040 arrested human cancer cells cycle in the G₁ phase of the cell cycle. The mechanism of this inhibition, which was demonstrated in non-small-cell lung cancer cells is believed to be through the inhibition of retinoblastoma protein (pRb) (Fukuoka, et al. (2000) Int J Cancer 88(5):810-819). Consequently, Aragusterol A is of utility in blocking cell cycle progression and inhibiting the retrogenic process in AAMI, MCI, AD, CVD and other retrogenic dementias.

Valproate (also known as valproate sodium, Depacon, Depakene, or valproic acid) is a compound which is approved in the form of sodium valproate for the treatment of seizure disorders. Valproate has been shown to induce cell cycle arrest in glioma cell lines. The mechanism which has been suggested for this effect is increased expression of cyclin D3 in the G₁ phase (Bacon et al. (2002) J Neurochem 83(1):12-19). This early phase cell cycle inhibiting effect indicates the utility of valproate in inhibiting the retrogenic process.

N-(3-chloro-7-indolyl)-1,4-benzenedisulfonamide (E7070) has been shown to block cell cycle progression of leukemia cells in the G₁ phase and therefore would be useful in treating retrogenic processes (Owa et al. (1999) J Med Chem 42 (19):3789-3799). The mechanism of this effect may be from suppression of cdk 2 and cyclin E activity (Owa et al.(2001) Curr Med Chem 8(12) :1487-1503).

As noted above in the reference to minocycline, p21 ras is associated with cell cycle activation (Lander et al. (1995) J. Biol. Chem. 270: 7017-7020). The farnesyl transferase inhibitors R115777, SCH66336 and BMS-214662 have been shown to prevent p21 ras activation and consequently, activation of the cell cycle (Owa, et al. (2001) Curr Med Chem 8(12) :1487-1503). Consequently, these compounds are of utility in inhibiting cell cycle progression and treating the retrogenic process in AAMI, MCI, AD, CVD, and other retrogenic dementias.

Another substance which has been shown to inhibit early phase cell cycle progression is sodium butyrate. This compound inhibits the expression of late G₁ phase genes such as cdc 2 (Charollais (1990) J Cell Physiol 145(1) :46-52). Consequently, the inventor concludes that sodium butyrate will be useful in inhibiting retrogenic processes.

General Inhibitors of Mitogenesis

In another embodiment, the second agent is a general mitogenic inhibitor. Preferably, the mitogenic inhibitor is flavopiridol, ciclopirox, or methotrexate. Inhibitors of Glutamate-Induced Excitotoxicity

In another embodiment the second agent or the third agent is an agent which reduced mitogenic stimulation by inhibiting glutamate-induced excitotoxicity. The inhibitor of glutamate-induced excitotoxicity is preferably memantine, neuramexane, amantadine (Symmetrel), riluzole, MK801, ketamine (Ketalar), dextromethorphan (Delsym or Sulphen DM), dextrorphan, phencyclidine, or dexanabinol (HU-21 1). Such inhibitors are preferably administered together with an early phase cell cycle inhibitor such as sirolimus.

Inhibitors of Activated Microglia

In yet another embodiment, the second agent or third agent is an inhibitor of an activated-microglial-related mitogenic factor, such as an anti-inflammatory agent. The anti-inflammatory agent is preferably selected from the group consisting of non-steroidal anti-inflammatory agents (NSAIDs), salicylates, steroids, and immunophillins. The NSAIDs are preferably selected from ibuprofen, naproxen, celecoxib, rofecoxib, sulindac, piroxicam, indomethacin, etodolac, nabumetone, tolmetin, diclofenac, ketoprofen, apazone, and meloxicam. In another embodiment, the steroid may be a glucocorticoid, for example, such as prednisone. In yet another embodiment, the immunophillin may be cyclosporine A or tacrolimus.

Combination of Early Cell Cycle and Mitogenic Stimulatory Inhibitors

The effects of the cell cycle blockers on preventing the clinical symptoms and progression of AAMI, MCI, AD, CVD and other retrogenic dementias can be enhanced by the simultaneous reduction of stimulatory factors which are known to promote cell cycle progression. For example, glutamate induced excitotoxicity is a known contributor to neurodegeneration (Orrego et al. (1993) Neuroscience 56:539-555; Lipton et al. (1994) New Engl. J. Med. 330:613-622). One agent which reduces this excitotoxicity is memantine. Memantine is an uncompetitive NMDA receptor antagonist ( Reisberg, et al. (2003) N. Engl. J. Med. 348:1333-1341). Other examples of uncompetitive NMDA receptor antagonists which reduce glutamate induced excitotoxicity include amantadine (Symmetrel) and neramexane (Danysz and Parsons (2002) Neurotox Res. 4:119-126; Rogoz, et al. (2002) Neuropharmacology 42:1024-1030) and dexanabinol (H U-211) (Biegon and Joseph (1995) Neurol Res. 17(4): 275-280) as well as ketamine (Ketalar), dextromethorphan (Delsym or Silphen DM), dextrorphan and phencyclidine (Parsons et al. (1995) Neuropharmacology 34:1239-1258). Memantine has recently been found to be useful for the treatment of moderate to severe AD (Reisberg et al. (2002) Neurobiol. Aging 23(1S): S555; Reisberg, et al. (2003) N. Engl. J. Med. 348:1333-1341) and also for the treatment of vascular dementia (Winblad et al. (1999) Int. J. Geriat. Psychiatry 14:135-146). Studies have shown that a potent inducer of excitotoxic effects, kainic acid, upregulates cyclin D1. A mitogenic inhibitor, flavopiridol, blocks exitotoxic neural death. Additionally an exitotoxicity blocker, MK801, also blocks neuronal death. The combination of the mitogenic inhibitor with the exitotoxicity blocker fully protects against neuronal death (Park et al. (2000) Neurobiol. Aging 21:771-781).

Another kind of blocker of excitotoxicity produced by glutamatergic activity is riluzole. Riluzole is known to be a sodium ion channel blocker with antiglutamatergic activity (Araki ,et al. (2001) Brain Res. 918(1-2):176-181; Mohammadi et al., (2002) Muscle Nerve 26(4):539-545). Riluzole can be used to reduce excitotoxicity induced cell cycle progression. Therefore, riluzole is therapeutic, alone and/or in combination with a cell cycle progression inhibitor in treating retrogenic processes including AAMI, MCI, AD, CVD, and other retrogenic dementias.

Another example of a mechanism which acts extracellularly to stimulate cell cycle factors and thereby stimulate the progression of retrogenic dementias is the production of activated microglia, which have been found to secrete mitogens (Wu et al. (2000) Neurobiol. Aging 21:797-806). One study found that β-amyloid alone and microglial cells alone did not produce an increase in cyclin D and related factors in labeled neurons. In contrast, amyloid labelled microglia did produce an increase in cyclin D and related cell cycle markers in labelled neurons (Wu et al. (2000) supra). The implication of this result is that only β-amyloid activated microglial cells elaborate mitogenic factors that trigger cell cycle-related neuronal death.

Counterintuitive Considerations in Familial Alzheimer's Disease

Although these studies support the inventor's realization that neuronal cell cycle inhibitors can retard the progression of AAMI, MCI, AD, CVD and related retrogenic dementias, the clinical application of this realization is much more complex. An indicator of this complexity comes from findings in familial AD (FAD) models. FAD disorders occur much earlier in life than the common late onset form of AD, and are associated with the presenilin genes. Two major forms of the presenilin FADs have been identified, presenilin1 (PS1) and presenilin 2 (PS2). These major forms of the presenilin dementias are associated with subtypes which are determined by the specific gene loci of the mutations. Initial findings indicate that presenilin overexpression in growing cell cultures resulted in arrest in the G₁ phase of the cell cycle and that the PS2 (N1411) mutant potentiated the cell cycle arrest (Janicki et al. (1999) Am. J. Pathol. 155:135-144). Further studies have demonstrated that in addition to the PS2 mutant, three different PSI mutations also increased cell cycle arrest (Janicki et al. (2000) Neurobiol. Aging 21:829-836). Of the three different PS1 mutations studied, PS1 (P117L), PS1 (P267S) and PS1 (E280A), the mutations which appear to be most aggressive in producing cell cycle arrest are associated with an earlier age of dementia onset, and the degree of cell cycle arrest appears to be hierarchically related to the age of onset. Specifically, the mutation associated with the greatest magnitude of cell cycle arrest, PS1 (P117L) has an age of onset as early as 27 years. In contrast the PS1 (E280A) mutation, which was the least associated with G₁ cell cycle arrest has an age of onset after approximately 45 years.

Methods of Treating Retrogenic Neurological Disorders and Methods of Administration

The invention provides methods of treatment comprising administering to a subject an effective amount of an inhibitor capable of inhibiting neuronal cell cycle progression, including inhibitors capable of inhibiting cell cycle progression prior to entry of a neuronal cell into a synthesis (S) phase. In a preferred aspect, the inhibitor compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In one specific embodiment, a non-human mammal is the subject. In another specific embodiment, a human mammal is the subject.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers or co-polymers such as Elvax (see Ruan et al. (1992) Proc. Natl. Acad. Sci. USA 89:10872-10876). In one embodiment, administration can be by direct injection by aerosol inhaler.

In another embodiment, the inhibitor compound can be delivered in a vesicle, in particular a liposome (see Langer (1990) Science 249:1527-1533; Treat et al. (1989) in Liposomes in the Therapy of Infectious Disease and Cancer, Lopez-Berestein and Fidler (eds.), Liss, New York, pp. 353-365).

In yet another embodiment, the inhibitor compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger et al. (1983) Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the airways, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

The present invention also provides pharmaceutical compositions for the treatment of AAMI, MCI, AD, CVD and related retrogenic dementias. Such compositions comprise a therapeutically effective amount of an agent capable of inhibiting neuronal cell cycle progression, preferably prior to entry of a neuronal cell into a synthesis (S) phase, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutic compounds useful in the method of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound useful in the method of the invention which will be effective in the treatment of AAMI, MCI, AD, CVD and related retrogenic dementias, can be determined by standard clinical techniques based on the present description. In addition, in vitro assays may optionally be employed to help identify optimal dosage ranges. The precise dose to be employed in the formulation will also depend on the route of administration, and the seriousness of the condition, disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

Treatment Population

Methods for identifying a population at risk for development of a retrogenic disorder are known to the art, including, for example, the global deterioration scale (GDS) (Reisberg et al. (1982) Am. J. Psychiatry 139: 1136-1139), the functional assessment staging procedure (FAST) (Reisberg (1988) supra), or the brief cognitive rating scale (Axes I to XI) (Reisberg et al. (1983a) Psychopharmacology Bulletin 19: 47-50; Reisberg et al. (1983b) Psychopharmacology Bulletin 19: 702-708; Reisberg et al. (1985) In: Senile Dementia of the Alzheimer Type (Traber & Gispen, eds.); Berlin: Springer-Verlag; pp. 18-37; Reisberg et al. (1992) In: Neurodevelopment, Aging and Cognition (Kostovic et al., eds.); Boston: Birkhäuser; pp. 345-369). A subject diagnosed as being in GDS stage 2, also termed “age associated memory impairment” (AAMI), age associated cognitive decline (Levy (1994) International Psychogeriatrics 6(1): 63-68), age related cognitive decline (APA (1994) Diagnositc and Statistical Manual of Mental Disorders, (4 ed.) Washington, D.C.: American Psychiatric Association), and/or age associated cognitive impairment, may be treated by the method of the invention to ameliorate the impairment and/or inhibit the progression of the condition. A subject identified at risk for development of AAMI is also a candidate for treatment by the method of invention for prevention of development of a degenerative condition. Subjects diagnosed as having more severe neurological or retrogenic conditions, such as MCI, AD, and CVD, may also be treated by the method of the invention to inhibit disease progression and/or ameliorate the degenerative condition.

What is claimed:

1. A therapeutic method of treating age associated memory impairment (AAMI), mild cognitive impairment (MCI), Alzheimer's disease (AD), cerebrovascular dementia (CVD) and related retrogenic degenerative neurological conditions, comprising administering a therapeutically effective amount of at least one agent capable of inhibiting a neuronal cell cycle progression.

2. The therapeutic method of claim 1, wherein the at least one agent is capable of inhibiting neuronal cell cycle progression before entry of a neuronal cell into a synthesis (S) phase.

3. The therapeutic method of claim 1, wherein the at least one agent is capable of inhibiting neuronal cell cycle progression at or prior to the early growth (G₁) phase.

4. The therapeutic method of claim 1, wherein the at least one agent is selected from the group consisting of minocycline, any tetracycline family derivative capable of crossing the blood brain barrier, acetylsalicylic acid, any salicylate which inhibits early phase cell cycle progression, sirolimus, any sirolimus derivative capable of inhibitng early cell cycle progression, flavopiridol, ciclopirox, a paulone, indirubin, fascaplycin, olomoucine, roscovitine, Aragusterol A, valproate, N-(3-chloro-7-indolyl)-1,4-benzenedisulfamide, a farnesyl transferase inhibitor such as R115777, SCH66336 and BMS-214662, and sodium butyrate.

5. A method of treating age associated memory impairment (AAMI), mild cognitive impairment (MCI), Alzheimer's disease (AD), cerebrovascular dementia (CVD) and related retrogenic degenerative neurological conditions, comprising administering a therapeutically effective amount of (i) at least one first agent capable of inhibiting neuronal cell cycle division before entry of a neuronal cell into an early phase or said cell cycle, and optionally (ii) at least one second agent capable of inhibiting cell cycle progression at any one or more of the phases of the cell cycle.

6. The method of claim 5, wherein the at least one first agent is selected from the group consisting of minocycline, any tetracycline family derivative capable of crossing the blood brain barrier, acetylsalicylic acid, any salicylate which inhibits early phase cell cycle progression, sirolimus, any sirolimus derivative capable of inhibiting early cell cycle progression, a paulone, indirubin, fascaplycin, olomoucine, roscovitine, Aragusterol A, valproate, N-(3-chloro-7-indolyl)-1,4-benzenedisulfamide, a farnesyl transferase inhibitor such as R115777, SCH66336 and BMS-214662, and sodium butyrate.

7. The method of claim 5, wherein the at least one second agent is selected from the group consisting of flavopiridol, ciclopirox, and methotrexate.

8. A method of treating age associated memory impairment (AAMI), mild cognitive impairment (MCI), Alzheimer's disease (AD), cerebrovascular dementia (CVD) and related retrogenic degenerative neurological conditions, comprising administering a therapeutically effective amount of:
i) at least one first agent capable of inhibiting neuronal cell cycle progression at or before an early phase;
ii) at least one second agent capable of inhibiting neuronal cell cycle progression generally; and optionally
iii) at least one third agent capable of inhibiting mitogenic stimulation.

9. The method of claim 8 wherein the first agent is selected from the group consisting of minocycline, any tetracycline family derivative capable of crossing the blood brain barrier, acetylsalicylic acid, any salicylate which inhibits early phase cell cycle progression, sirolimus, any sirolimus derivative capable of inhibitng early cell cycle progression, a paulone, indirubin, fascaplycin, olomoucine, roscovitine, Aragusterol A, valproate, N-(3-chloro-7-indolyl)-1,4-benzenedisulfamide, a farnesyl transferase inhibitor such as R115777, SCH66336 and BMS-214662, and sodium butyrate.

10. The method of claim 8, wherein the second agent is selected from the group consisting of flavopiridol, ciclopirox, and methotrexate.

11. The method of claim 8 wherein the third agent acts to inhibit glutamate-induced excitotoxicity and/or activated microglia-induced mitogenic stimulation.

12. The method of claim 11, wherein the inhibitor of glutamate-induced excitotoxicity is selected from the group consisting of memantine, neramexane, amantadine, riluzole, MK801, ketamine, dextromethorphan, dextrorphan, phencyclidine, and dexanabinol (HU-211).

13. The method of claim 11, wherein the inhibitor of activated microglia-induced mitogenic stimulation is an anti-inflammatory agent.

14. The method of claim 13, wherein the anti-inflammatory agent is selected from the group consisting of non-steroidal anti-inflammatory agents (NSAIDS), salicylates, steroids, and immunophillins.

15. The method of claim 14, wherein the NSAID is selected from the group consisting of ibuprofen, naproxen, celecoxib, rofecoxib, sulindac, piroxicam, indomethacin, etodolac, nabumetone, tolmetin, diclofenac, ketoprofen, apazone, and meloxicam.

16. The method of claim 14, wherein the steroid is a glucocorticoid.

17. The method of claim 16, wherein the glucocorticoid is prednisone.

18. The method of claim 14, wherein the immunophillins is selected from the group consisting of cyclosporine A and tacrolimus.

19. The method of claim 5, wherein the first agent is capable of inhibiting neuronal cell cycle progression at, or prior to entry into, the early growth (G₁) phase.

20. The method of claim 5, wherein the first agent is capable of inhibiting neuronal cell cycle progression at, or prior to entry into, the synthesis (S) phase.

21. The method of claim 20, wherein the agent is selected from the group consisting of minocycline, any tetracycline family derivative capable of crossing the blood brain barrier, acetylsalicylic acid, any salicylate which inhibits early phase cell cycle response, sirolimus, any sirolimus derivative capable of inhibiting early cell cycle progression, a paulone, indirubin, fascaplycin, olomoucine, roscovitine, Aragusterol A, valproate, N-(3-chloro-7-indolyl)-1,4-benzenedisulfamide, a farnesyl transferase inhibitor such as R115777, SCH66336 and BMS-214662, and sodium butyrate.

22. The method of claim 1, wherein the subject is a human.

23. A method of treating age associated memory impairment (AAMI), mild cognitive impairment (MCI), Alzheimer's disease (AD), or cerebrovascular dementia (CVD), in a subject with AAMI, MCI, AD, or CVD, comprising administering a therapeutically effective amount of (i) at least one first agent capable of inhibiting neuronal cell cycle progression, and (ii) at least one second agent capable of reducing mitogenic stimulation.

24. The method of claim 23, wherein the at least one first agent inhibits cell cycle progression prior to entry of a neuronal cell into a synthesis (S) phase and the at least one second agent is capable of inhibiting glutamate-induce excitotoxicity and/or reducing activated microglia-induced mitogenic stimulation.

25. The method of claim 23, wherein the at least one first agent inhibits cell cycle progression at or prior to entry of a neuronal cell into, an early growth (G₁) phase and the at least one second agent is capable of inhibiting glutamate-induce excitotoxicity and/or reducing activated microglia-induced mitogenic stimulation.

26. The therapeutic method of claim 23, wherein the at least one first agent is selected from the group consisting of minocycline, any tetracycline family derivative capable of crossing the blood brain barrier, acetylsalicylic acid, any salicylate which inhibits early phase cell cycle progression, sirolimus, any sirolimus derivative capable of inhibiting early cell cycle progression, a paulone, indirubin, fascaplycin, olomoucine, roscovitine, Aragusterol A, valproate, N-(3-chloro-7-indolyl)-1,4-benzenedisulfamide, a farnesyl transferase inhibitor such as R115777, SCH66336 and BMS-214662, and sodium butyrate.

27. The method of claim 23 wherein the second agent acts to inhibit glutamate-induced excitotoxicity and/or activated microglia-induced mitogenic stimulation.

28. The method of claim 27, wherein the inhibitor of glutamate-induced excitotoxicity is selected from the group consisting of memantine, neramexane, amantadine, riluzole, MK801, ketamine, dextromethorphan, dextrorphan, phencyclidine, and dexanabinol (HU-211).

29. The method of claim 27, wherein the inhibitor of activated microglia-induced mitogenic stimulation is an anti-inflammatory agent.

30. The method of claim 29, wherein the anti-inflammatory agent is selected from the group consisting of non-steroidal anti-inflammatory agents (NSAIDS), salicylates, steroids, and immunophillins.

31. The method of claim 30, wherein the NSAID is selected from the group consisting of ibuprofen, naproxen, celecoxib, rofecoxib, sulindac, piroxicam, indomethacin, etodolac, nabumetone, tolmetin, diclofenac, ketoprofen, apazone, and meloxicam.

32. The method of claim 30, wherein the steroid is a glucocorticoid.

33. The method of claim 32, wherein the glucocorticoid is prednisone.

34. The method of claim 30, wherein the immunophillins is selected from the group consisting of cyclosporine A and tacrolimus.

35. The method of claim 23 wherein the at least one first agent is selected from the group consisting of flavopiridol, ciclopirox, and methotrexate and the at least one second agent acts to inhibit glutamate-induced excitotoxicity and/or activated microglia-induced mitogenic stimulation.
